(19) United States
(12) Patent Application Publication
(10) Pub. No.: US 2017/0216340 A1
(43) Pub. Date: Aug. 3, 2017

(54) A Nucleoside Derivative For Use As A Drug, Particularly For The Treatment Of Chronic Lymphocytic Leukemia

(71) Applicant: INSTYTUT BIOLOGII MEDYCZNEJ POLSKIEJ AKADEMII NAUK, Lodz (PL)

(72) Inventors: Zbigniew Lesnikowski, Lodz (PL); Agnieszka Olejniczak, Lodz (PL); Zofia Kilianska, Lodz (PL); Jolanta Zolnierczyk, Lodz (PL); Tadeusz Robak, Lodz (PL); Adam Mieczkowski, Warszawa (PL)

(21) Appl. No.: 15/501,669

(22) PCT Filed: Aug. 5, 2015
(86) PCT No.: PCT/IB2015/055945

§ 371 Date: Feb. 3, 2017

(30) Foreign Application Priority Data
Aug. 5, 2014 (IT) RM2014A000459

(51) Int. Cl. A61K 31/7076; C07H 23/00

(52) U.S. Cl. CPC A61K 31/7076; C07H 23/00

(57) ABSTRACT

We disclose nucleoside derivatives useful as drugs, particularly for the treatment of chronic lymphocytic leukemia.

The subject of the present invention is a derivative of an antileukemic drug, preferably against chronic lymphocytic leukemia (CLL).

The currently available antileukemic drugs have several drawbacks that narrow their clinical utility including limited effectiveness, often sever side effects and emergence of drug-resistant cancer variants possessing an increasing problem for disease management. Thus, there is still a strong need to identify new targets for antileukemic chemotherapy and to develop novel anticancer compounds and treatment strategies.

Subject of the invention has been defined in the attached claims. Example embodiments of the present invention have been described in the following examples and are shown in the attached figures which Scheme 2 shows synthesis of 2-ethynyl-(1,12-dicarb a-closo-dodecaboran-2-yl)adenosine (3) modified with para-carborane cluster in the Sonogashira-type reaction between 2-iodoadenosine and 2-ethynyl-1, 12-dicarba-closo-dodecaborane (2) catalyzed by palladium tetrakis(triphenylphosphine)palladium(0). Scheme 3. shows synthesis of 8-ethynyl-(1, 12-dicarba-closo-dodecaboran-2-yl)-2′-deoxyadenosine (5) obtained according similar procedure as for 3, between 8-bromo-2′-deoxyadenosine (4) and boron cluster donor (2) All of the described example compounds according to the present invention and intermediate compounds have been fully characterized by UV, IR, ¹H-, ¹³C-, ³¹P- and ¹¹B-NMR, FAB-MS and chromatographic methods.

The biological activity of compounds according to the present invention was evaluated by determination their cytotoxicity, their ability to induce apoptosis in tumour cells, their ability to activate caspase 3 and to influence the protein profile of tumour cells evaluated using Western blotting. The results of the analyses performed are summarised below and described in detail in the following examples.

### Cytotoxicity and Apoptosis Induction

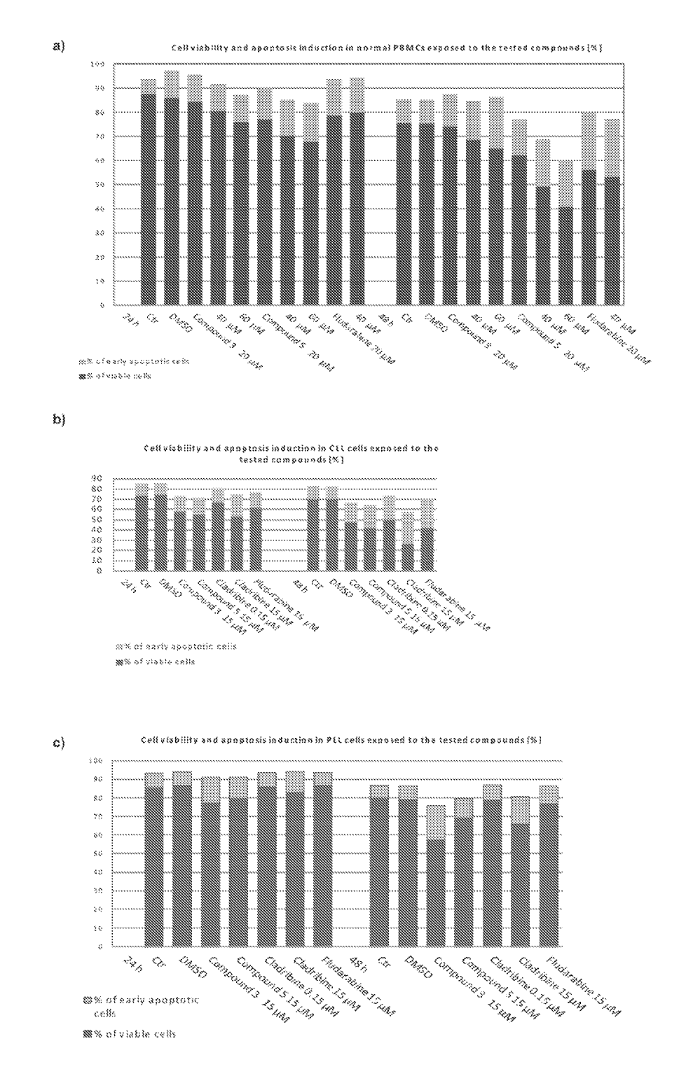

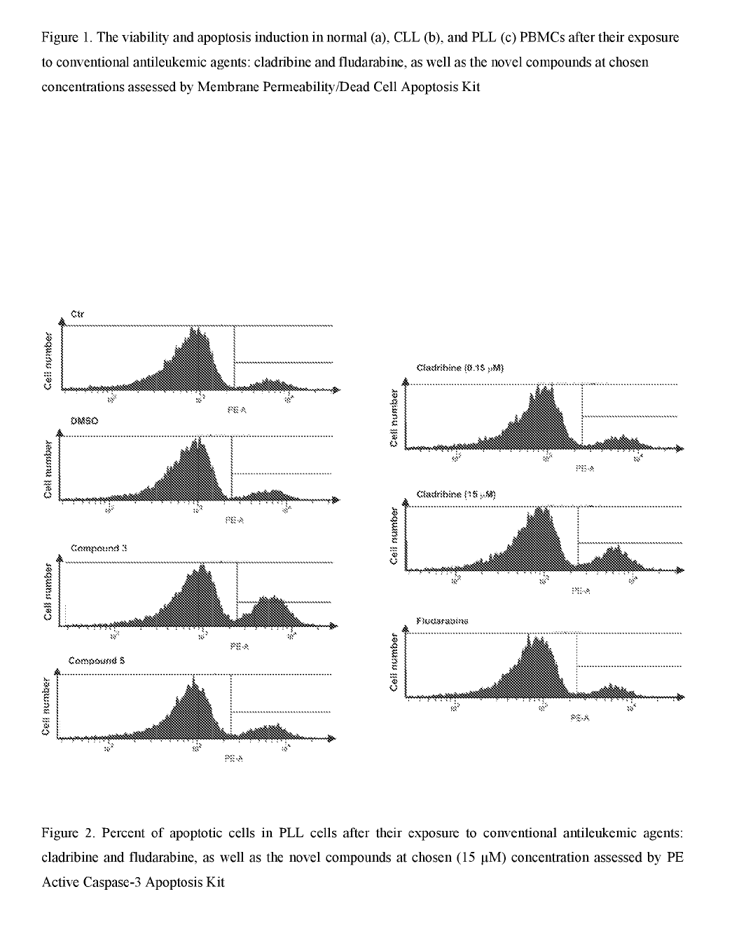

During the first stage, the cytotoxicity analysis of compounds according to the present invention introduced into the culture medium in increasing concentrations (from 0 μM to 60 μM) against leukemic and healthy peripheral blood mononuclear cells (PBMCs). The goal of this stage was to select the optimal concentrations which would be useful for the further comparative analysis of novel compound with conventional chemotherapeutics, i.e. cladribine and fludarabine, in terms of their antileukemic efficacy. The cytotoxicity of compounds was evaluated in PBMCs isolated from the blood of 5 patients with CLL, 1 with PLL and 5 healthy donors after 24 and 48 h. of incubation. Cell survival was evaluated cytometrically using propidium iodide staining using the Membrane Permeability/Dead Cell Apoptosis assay (propidium iodide+YO-PRO). The reference for compounds according to the present invention were PBMCs exposed to DMSO at a concentration corresponding to that introduced into the culture environment along with the increasing concentration of the tested compounds. The reference for the cladribine and fludarabine were cells incubated solely in complete culture medium (control). FIG. 1a represents the survival of healthy cells treated with compounds according to the present invention at increasing concentrationsand evaluated using the Membrane Permeability/Dead Cell Apoptosis assay.

Healthy cells showed a low sensitivity to the newly obtained compounds administered at concentrations below 20 μM. In contrast to these, the leukemic PBMCs occurred to be highly sensitive for the novel nucleoside derivatives. Moreover, the differences between the cytotoxicity of C-8 (compound 5) and C-2 (compound 3) modified adenosine were observed. Compound 5 seemed to be more cytotoxic in comparison to compound 3 The latter showed smaller anti-leukemic potential against CLL cells than compound 5. Importantly, compound 3 was less cytotoxic against normal PBMCs at the same time. Moreover, the cytotoxicity of compound 3 against normal cells was lower than fludarabine.

The 15 μM concentration of the used agents, as safe for normal PBMCs, was selected for further analyses with regard to their apoptosis-inducing potential. The viability and apoptosis induction were examined in cell samples obtained from peripheral blood of 6 patients with leukemia (5 cases of CLL and 1 case of PLL). These results are shown in FIGS. 1b and 1c. Both of newly synthesized agents revealed high pro-apoptotic potential against CLL cells comparable to cladribine at clinically administered concentration (0.15 μM) and fludarabine. It is worth noting that the agents differed in their apoptosis inducing potential in PLL cells. Our very preliminary data shown that compound 3 seemed to be very potent against PLL cells, very rare hematological cancer. Anti-leukemic potential of the compound 3 against these cells was even higher than that of cladribine and fludarabine.

### Caspase 3 Activation

Caspase 3 is a main effector caspase which is involved in apoptosis process realization (Miura M., Zhu H., Otello R., Hartwieg E.A., Yuan J. 1993. Induction of apoptosis in fibroblasts by IL-1 beta-converting enzyme, a mammalian homolog of the C. elegans cell death gene ced-3, Cell, 75, 653-660). In the present investigation we revealed the increase of number of cells with caspase 3 active form among PLL cells treated with tested agents. Our analysis, conducted after 48 h drug exposure, confirmed that in PLL cells a high antileukemic potential of adenosine modified at C-2 position with boron cluster is related to apoptosis induction. The number of cells with active form of the enzyme raised after the exposure to compound 3 from 9.4% and 10.6% in control and vehicle control (DMSO), respectively, to 28.5%. Similar effect (20,1%) was observed after the incubation of PLL cells with cladribine at concentration of 15 μM which recursively exceeds its level in serum of patients during antileukemic therapy. By contrast, the number of caspase-3-positive cells among PLL cells incubated with 0.15 μM cladribine and fludarabine was only slightly increased (11.8% in both cases). Of note, compound 5 affected PLL cells weaker than compound 3 which was confirmed by caspase 3 activation in 16.1% of leukemic cells.

### Western Blot Analysis

The expression of apoptosis-related proteins was assessed in CLL and PLL cells exposed to the tested agents. All examined adenosine derivatives triggered proteolysis of PARP-1 full length protein (116 kDa) to its cleavage product (89 kDa), which is a known marker of apoptosis. Moreover, altered expression of pro-apoptotic (Bax) and anti-apoptotic proteins (Mcl-1, Bcl-2) from Bcl-2 family was also observed in purine derivative-treated leukemic cells. However, there were some individual differences between basal levels of these apoptotic regulators in model cells, as well as the changes in their cellular levels after the agent exposure were diverse. For instance, there were no significant alterations in Bax/Bcl-2 ratio in cell samples obtained from exemplary patient 1 exposed to the novel nucleosides. While, an essential increase of Bax expression level and Bcl-2 protein diminution were seen in cladribine and fludarabine-treated cells. On the other hand, in PBMC samples from Patient 2 a clear decrease of the expression level of Bcl-2 (in comparison to vehicle control) was observed only for these cells which had been exposed to compound 5 or 3.

### EXAMPLE 1

### Cytotoxicity and Apoptosis Assay

PBMC cells were isolated from peripheral blood using centrifugation via a Histopaque gradient according to the manufacturer's instructions. Next, PBMC samples were cultured in RPMI 1640 medium supplemented with heat-inactivated 10% fetal bovine serum, L-glutamine, and antibiotics (streptomycin 100 μg/mL, penicillin 100 U/mL) at 37° C., 5% CO₂, fully humidified atmosphere. For cytotoxicity evaluation the leukemic cells were incubated in culture medium without tested compounds (control) or were exposed to DMSO (vehicle control) at concentrations of 0.08%-0.4%, as well as to cladribine, fludarabine, and adenosine modified with boron cluster at C-8 (compound 5) and C-2 (compound 3) positions of purine ring; all at concentrations of 0.1-60 μM. The cytotoxicity was assessed cytometrically after 24 and 48 h incubation.

Firstly, it was analyzed by propidium iodide staining. Then, Membrane Permeability/Dead Cell Apoptosis Kit (Molecular Probes, Invitrogen™) was used to examine the contribution of apoptosis to cell death induction after exposure to the used agents. The analyses were performed on BD LSR II flow cytometry using FACSDiva Version 6.1.2 software. Ten thousand events were examined for each analysis. The distinction between living (YO-PRO-negative/propidium iodide-negative), early apoptotic (YO-PRO-positive/propidium iodide-negative) and dead (YO-PRO-positive/propiodium iodide-positive) cells was based on the differences in their permeability for fluorescent dyes. The results are shown as the percent of viable and early apoptotic cells in gated PBM cell population, respectively.

The cytotoxicity of normal PBMCs (isolated from volunteers without leukemia) was evaluated using the same test after 24 and 48 h exposure to the examined agents at the concentrations of 20 μM, 40 μM, and 60 μM.

For further analyses the concentration of 15 μM of all tested agents was chosen. In addition, 0.15 μM concentration of cladribine was used, which corresponds to cladribine concentration in serum of leukemic patients during the anticancer therapy.

Cladribine (Biodrybina) and fludarabine were purchased from the Institute of Biotechnology and Antibiotics Bioton (Warsaw, Poland) and Schering AG (Berlin, Germany), respectively. Adenosine derivatives modified with boron clusters were synthesized by Prof. Zbigniew J. Leśnikowski (Institute for Medical Biology of the Polish Academy of Sciences, Lodz, Poland) (see results in FIG. 1) and Institute of Biochemistry and Biophysics of the Polish Academy of Sciences (Dr. Adam Mieczkowski, compounds 7 and 8). FIG. 1 shows the viability and apoptosis induction in normal (a), CLL (b), and PLL (c) PBMCs after their exposure to conventional antileukemic agents: cladribine and fludarabine, as well as the novel compounds at chosen concentrations (15 μM, in the case of cladribine, also 0.15 μM) assessed by Membrane Permeability/Dead Cell Apoptosis Kit

### EXAMPLE 2

### Caspase-3 Activation Assay

The percent of leukemic PBMCs with active form of caspase-3 was measured cytometrically by PE Active Caspase-3 Apoptosis Kit, BD Pharmingen according to manufacturer's protocol. The analyses were conducted after 48 h incubation with/without (control) the agents or DMSO (vehicle control). BD LSR II flow cytometer was used and the analyses were performed using FACSDiva Version 6.1.2 software (see results in FIG. 2). FIG. 2 shows Percent of apoptotic cells in PLL cells after their exposure to conventional antileukemic agents: cladribine and fludarabine, as well as the novel compounds at chosen (15 μM) concentration assessed by PE Active Caspase-3 Apoptosis Kit.

### EXAMPLE 3

### Western Blot Analysis

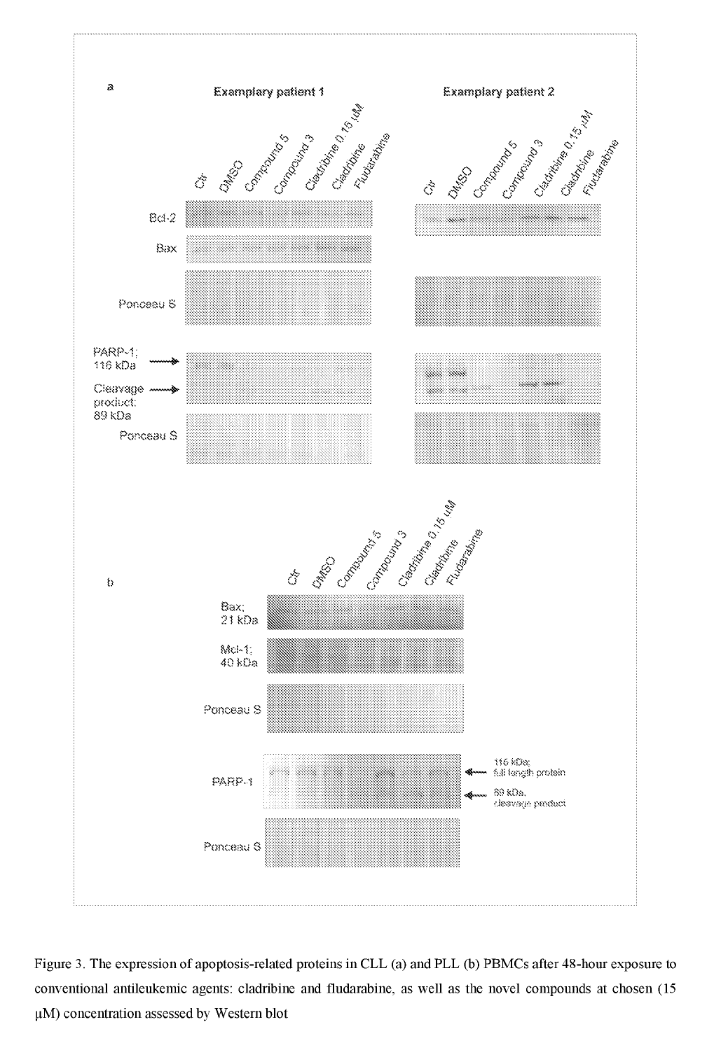

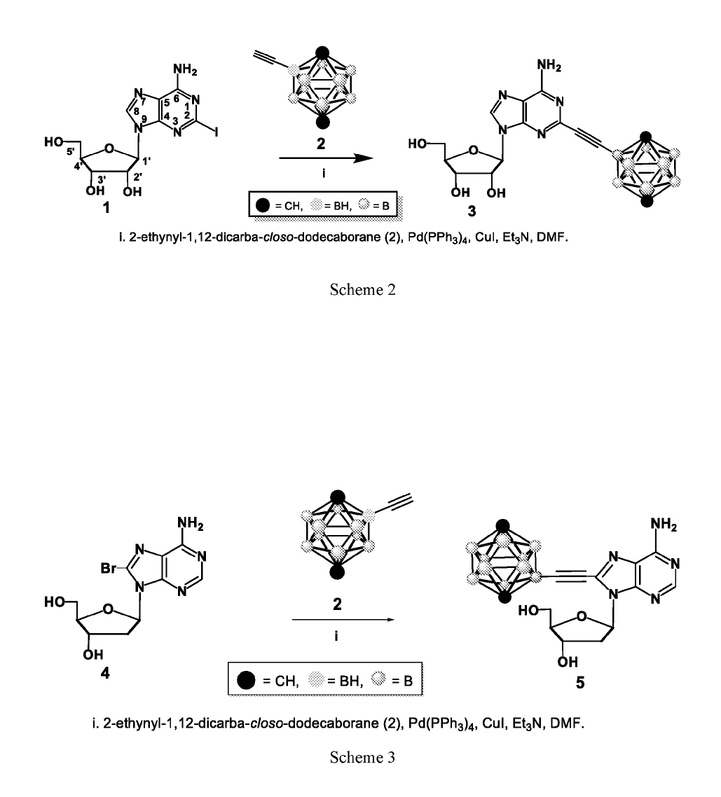

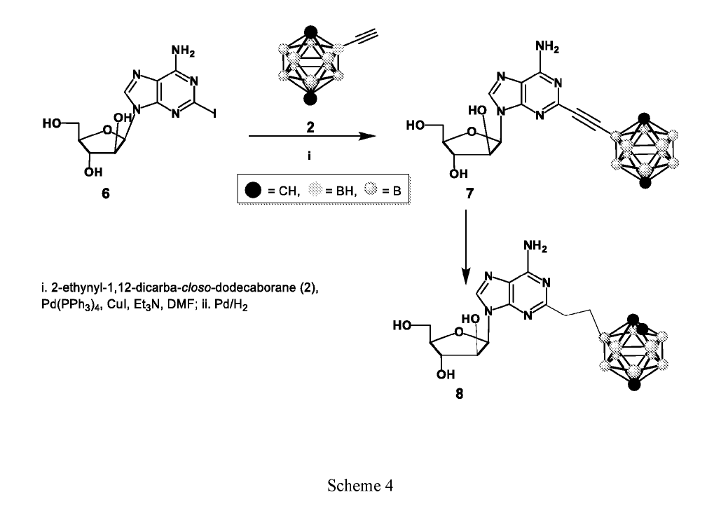

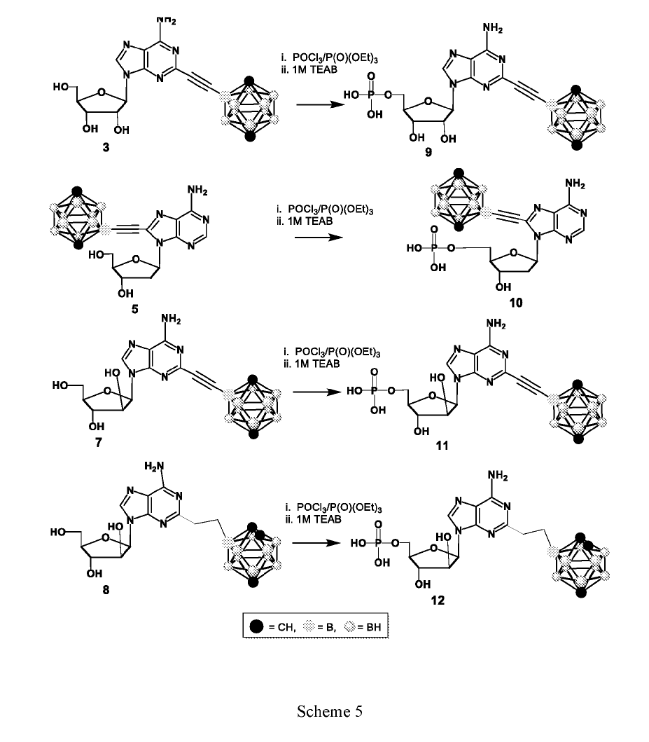

Control PBMCs (without the agents), as well as the cells samples exposed to DMSO (vehicle control) and the tested agents were lysed (4° C., 1 h) in a buffer containing 10 mM Tris-HCl (pH 7.5), 300 mM NaCl, 1% Triton X-100, 2 mM MgCl₂, 0.1 M DTT, and protease inhibitors as described previously (A., Kobylinska, J., Bednarek, J. Z., Blonski, M., Hanausek, Z., Walaszek, H., Piekarski, T., Robak, Z. M., Kilianska. 2006. In vitro sensitivity of B-cell chronic lymphocytic leukemia to cladribine and its combinations with mafosfamide and/or mitoxantrone, Oncol. Rep., 16, 1389-1395). Protein content was estimated according to Lowry method (O. H., Lowry, N. J., Rosebrough, A. L., Farr, R. J., Randall. 1951. Protein measurement with the Folin phenol reagent. J. Biol. Chem., 193, 265-275) and the cell lysates were prepared for subsequent Western blotting analysis. Protein samples (60 μg/lane) were separated by SDS polyacrylamide gel electrophoresis (SDS-PAGE) on 8.0 or 12.5% slab gels and electrotransfered onto Immobilon P (H., Towbin, T., Staechlin, J., Gordon, 1979, Electrophoretic transfer of protein from polyacrylamide gels to nitrocellulose sheets: procedure and some applications. Proc. Natl. Acad. Sci. USA, 76, 4350-4354). Membrane staining with 0.5% Ponceau S solution was done to confirm equal protein loading and completeness of the transfer. The membranes were saturated in 5.0% skim milk in TBS (10 mM Tris-HC1, pH 7.5, 150 mM NaC1) for 1 h at ambient temperature, and then incubated overnight with antibodies specific to Mcl -1 (1:1000), Bcl-2 (1:1000), Bax (1:1000), PARP-1 (1:1000), all from Santa Cruz Biotechnology Inc. Antigen recognition was performed with appropriate secondary antibodies conjugated with horseradish peroxidase. The antigen-antibodies complexes were detected with Novex HRP Chromogenic substrate (TMB) from Invitrogen or, alternatively, by chemiluminescence method (see results in FIG. 3). FIG. 3 shows the expression of apoptosis-related proteins in CLL (a) and PLL (b) PBMCs after 48-hour exposure to conventional antileukemic agents: cladribine and fludarabine, as well as the novel compounds at chosen (15 μM) concentration assessed by Western blot.

### EXAMPLE 4

### Synthesis of 2-iodoadenosine, Compound 1

Synthesis of compound 1 was performed according to literature procedure (Robins, M. J.; Uznański, B. Nucleic Acid Related Compounds. 33. Conversions of Adenosine And Guanosine to 2,6-Dichloro, 2-Amino-6-Chloro, and Derived Purine Nucleosides. Can. J. Chem. 1981, 59, 2601-2606; Matsuda A., Shinozaki M., Yamaguchi T., Homma H., Nomoto R., Miyasaka T., Watanabe Y., Asiru T., Nucleosides and Nucleotides. 103. 2-Alkynyladenosines: A Novel Class of Selective Adenosine A2 Receptor Agonists with Potent Antihypertensive Effects, J. Med. Chem., 1992, 35, 241-252).

### EXAMPLE 5

### Synthesis of 8-bromo-2′-deoxyadenosine, Compound 4

Synthesis of compound 4 was performed according to literature procedure (Ikehara, M.; Kaneko, M. Studies of Nucleosides and Nucleotides. XLIV. Purine Cyclonucleosides. Synthesis of Cyclonucleosides having 8,3′-O- and -S-Anhydro Linkage derived from 2′-Deoxyadenosine. Chem. Pharm. Bull. 1970, 18, 2441-2446).

### EXAMPLE 6

### Synthesis of 2-iodo-arabinoadenosine, Compound 6

Synthesis of compound 6 was performed according to literature procedure ((Robins, M. J.; Uznański, B. Nucleic Acid Related Compounds. 33. Conversions of Adenosine And Guanosine to 2,6-Dichloro, 2-Amino-6-Chloro, and Derived Purine Nucleosides. Can. J. Chem. 1981, 59, 2601-2606; Matsuda A., Shinozaki M., Yamaguchi T., Homma H., Nomoto R., Miyasaka T., Watanabe Y., Asiru T., Nucleosides and Nucleotides. 103. 2-Alkynyladenosines: A Novel Class of Selective Adenosine A2 Receptor Agonists with Potent Antihypertensive Effects, J. Med. Chem., 1992, 35, 241-252).

### EXAMPLE 7

### Synthesis of 2-ethynyl-1,12-dicarba-closo-dodecaborane, Compound 2

Synthesis of compound 2 was performed according to the literature procedure (Jiang, W.; Knobler, C. B.; Curtis, C. E.; Mortimer, M. D.; Hawthorne, M. F. Iodination Recation of Icosahedral para-carborane and the Synthesis of Carborane Derivatives with Boron-Carbon Bonds. Inorg. Chem. 1995, 34, 3491-3498).

### EXAMPLE 8

### Synthesis of 2-ethynyl-(1,12-dicarba-closo-dodecaboran-2-yl)adenosine, Compound 3

Synthesis of compound 3 was performed according to the literature procedure (K. Bednarska, A. B. Olejniczak, A. Piskala, M. Klink, Z. Sulowska, Z. J. Leśnikowski, Effect of adenosine modified with a boron cluster pharmacophore on reactive oxygen species production by human neutrophils, Bioorg. Med. Chem., 2012, 20, 6621-6629).

This synthesis was performed under anhydrous conditions in an argon atmosphere. 2-lodoadenosine (1, 50 mg, 0.13 mmol), 2-ethynyl-1,12-dicarba-closo-dodecaborane (2, 23.56 mg, 0.14 mmol), CuI (3.06 mg, 0.016 mmol) and Pd(PPh₃)₄ (6.36 mg, 0.0055 mmol) were placed in a flame-dried flask and dissolved in anhydrous DMF (0.9 mL) and Et₃N (0.3 mL). The resultant solution was stirred for 75 min at room temperature and then for 70 min at 80° C. After the reaction was complete, the volatiles were evaporated under reduced pressure, and the residue was dissolved in ethyl acetate (12 mL). The resultant solution was extracted with deionised water (2×6 mL). The organic fractions were collected, washed with 0.5% aqueous EDTA (1×2 mL), dried over anhydrous magnesium sulphate and concentrated under reduced pressure. The crude product was purified by silica gel column chromatography (2 g, 230-400 mesh) using a linear gradient of CH₃OH in CH₂Cl₂ (0-10%) as the eluent. Yield: 37.68 mg (68%). TLC (CH₂Cl₂:CH₃OH, 9:1 v/v): R_{f}=0.11; UV-Vis (95% C₂H₅OH, nm): λₘᵢₙ=280, 251, λₘₐₓ=300, 270, 239; ¹H NMR ((CD₃)₂CO₃ ppm): δ=8.31, (s, 1H, H-8), 6.81 (bs, 2H, NH₂), 5.97 (d, 1H, ¹J_{1′:2′}=7.50, H-1′), 5.10-5.05 (m, 2H-5′, 1H—OH), 4.82-4.73 (m, 2H, 1H-2′, 1H—OH), 4.39-4.37 (m, 2H, 1H-3′, 1H—OH), 4.16-4.14 (m, 1H, H-4′), 4.16-4.14 (m, 1H, H-4′), 3.84-3.71 (m, 3H, 2H-5′, 5″, C(₁)H_{carborane}) , 3.51 (bs, 1H, c(12)H^{carborane}), 3.50-1.00 (m, 9H, BH^{carborane}); ¹³C NMR ((CD₃)₂CO, ppm): δ=157.02 (C-6), 150.00 (C-2), 146.21 (C-4), 142.15 (C-8), 121.00 (C-5), 90.38 (C-1′), 87.81 (C-4′), 75.17 (C-2′), 72.38 (C-3′), 67.53 (CH^{carborane}); 64.90 (CH^{carborane}), 63.20 (C-5′); ¹¹B NMR ((CD₃)₂CO, ppm): coupled δ=−12.18 (s, 2B), −13.90 (s, 4B), −15.90 (s, 4B); MS (FAB): m/z =435.40 (M+2)⁺, 497.4 (M+Na+K+2)⁺, C₁₄H₂₃B₁₀N₅O₄ [433.28].

### EXAMPLE 9

### Synthesis of 8-ethynyl-(1,12-dicarba-closo-dodecaboran-2-yl-2′-deoxyadenosine, Compound 5

Synthesis of compound 5 was performed according to the literature procedure (Bednarska K., Olejniczak A. B., Wojtczak B. A., Sulowska Z., Leśnikowski Z. J., Adenosine and 2′-deoxyadenosine modified with boron cluster pharmacophores as new classes of human blood platelet function modulators, ChemMedChem., 2010, 5, 749-756).

Nucleoside substrate, 8-bromo-2′-deoxyadenosine (4, 46.2 mg, 0.4 mmol) lyophilized from water (0.5 mL), 2-ethynyl-para-carborane (2, 25.2 mg, 0.15 mmol), cuprous iodide (3.2 mg, 0.017 mmol) and tetrakis(triphenylphosphine)palladium(0) (6.8 mg, 0.006 mmol) were placed in 25 mL burner-dried round bottom flask, then anhydrous dimethyloformamide (0.9 cm³) and triethylamine (0.3 mL) were added. The reaction was carried out under argon atmosphere, with stirring, at 80° C. After 30 min the reaction was quenched by evaporation of the solvent. Dichloromethane (10 cm³) was added to the residue then resultant solution was extracted with distilled water (2×5 mL). Organic layer was collected and extracted with 0.5% aqueous EDTA (2×5 mL) then dried over MgSO₄. Drying agent was filtered off and washed with dichloromethane. The organic fractions and washing were combined then solvent was evaporated under reduced pressure. Crude product 5 was purified by column chromatography on silica gel (4 g, 230-400 mesh); as an eluting solvent a linear gradient of CH₃OH in CH₂Cl₂ was used (0-14%). Yield: 41.5 mg (71%) as an opalescent, colorless oil: R_{f}=0.46 (CH₂Cl₂/CH₃OH, 1:9); RP-HPLC: Rₜ=20.08 min; ¹H-NMR (250.131 MHz, CD₃OD, Me₄Si): δ=0-4 (11H, BH-carborane), 2.2-2.4 (m, 1H, 2′-H), 3.03-3.09 (m, 1H, 2″-H), 3.62-3.69 (m, 2H, 5′-H, 5″-H), 3.85-3.88 (m, 1H, 4′-H), 4.43-4.45 (m, 1H, 3′-H), 6.39 (t, 1H, 1′-H, J³_{1′2′}=7.78, J³_{1′2′}=6.62), 8.15 (s, 1H, 2-H); ¹³C-NMR (62.90 MHz, CD₃OD, Me₄Si): δ=40.31 (C-2′), 64.16 (C-5′), 65.45, 67.90 (CH-carborane), 73.60 (C-3′), 88.12 (C-1′), 90.34 (C-4′), 120.77 (C-5), 134.90 (C-8), 149.45 (C-4), 154.31 (C-2), 157.45 (C-6); ¹¹B-NMR (80.20 MHz, CD₃OD, BF₃/Et₂O): δ=−12.99 (9H, BH); UV/Vis (EtOH): λₘᵢₙ=250 nm, λₘₐₓ=236, 301 nm; MS (CI, isobutane) m/z [M+1]⁺ calcd for C₁₄H₂₄B₁₀N₅O₃: 418.482, found: 419. 3.

### EXAMPLE 10

### Synthesis of 2-ethynyl-(1,12-dicarba-closo-dodecaboran-2-yl)arabinoadenosine, Compound 7

This synthesis was performed under anhydrous conditions in an argon atmosphere. 2-iodo-9-(β-D-arabinofuranosyl)adenine (6, 144.5 mg, 0.37 mmol), 2-ethynyl-1,12-dicarba-closo-dodecaborane (2, 75.3 mg, 0.45 mmol), CuI (8.8 mg, 0.046 mmol), Pd(PPh₃)₄ (18.4 mg, 0.016 mmol) were placed in the oven dried flask (130° C.) and dissolved in anhydrous DMF (2.6 mL) and Et₃N (0.9 mL). The resultant solution was stirred for 75 min at room temperature and then for 70 min at 80° C. After the reaction was complete, the solvents were evaporated under reduced pressure in 40° C. water bath and residue was dissolved in ethyl acetate 50 mL. The resultant solution was extracted with deionised water (2×20 mL). The organic fractions were collected, washed with 0.5% aqueous EDTA (1×10 mL), dried over anhydrous magnesium sulphate and concentrated under the reduced pressure. The crude product was purified by silica gel column chromatography (8 g, 230-400 mesh) using 10% methanol in chloroform as an eluent. Yield: 125.5 mg (79%). TLC (CH₂Cl₂/CH₃OH, 4:1): R_{f}=0.49, (CH₂Cl₂/CH₃OH, 9:1): R_{f}=0.18; ATR-FTIR (cm⁻¹): v=3500-3000 (OH, NH), 2953 (CH₂), 2919 (CH₂), 2850 (CH₂), 2614 (BH), 1655 (NH₂), 1590, 1504, 1454 (C═C^{arom}), 1042 (C—O); ¹H NMR (CD₃OD, ppm): δ=1.700-2.880 (m, 10H, B—H), 3.399, 3.646 (2s, 2H, C—H^{p-carborane}) 3.863-3.933 (qd, 2H, 5′-H), 3.998-4.019 (q, 1H, 4′-H), 4.298-4.336 (2t, 2H, 2′-H, 3′-H), 6.412-6.420 (d, 1H, l′-H), 8.437 (s, 1H, 8-H); ¹¹B{H} NMR (CD₃OD, ppm): δ=−14.89 (s), −14.16 (s), −13.25 (s); ¹³C{H} NMR (CD₃OD, ppm): δ=63.40 (C-5′), 66.13 (C^{p-carborane}) 68.91 (C^{p-carborane}), 77.95, 78.55 (C-2′, C-3′), 86.92, 87.38 (C-1′, C-4′), 120.42 (C-5), 144.24 (C-8), 147.86 (C-4), 151.57 (C-2), 157.94 (C-6); MS (FAB, Gly): m/z=433.3 [M], calc. for C₁₄H₂₃B₁₀N₅O₄=433.7.

### EXAMPLE 11

### Synthesis of 2-ethyl-(1,12-dicarba-closo-dodecaboran-2-aarabinoadenosine, Compound 8

2-Ethynyl-(1,12-dicarba-closo-dodecaboran-1-yl)-9-(β-D-arabinofuranosyl)adenine (7) (52.3 mg, 012 mmol) was dissolved in 10 mL of ethanol and 30 mg of palladium on activated charcoal (10% Pd basis) were added to the reaction mixture. The reaction vessel was connected to the balloon filled with hydrogen and reaction mixture was stirred in room temperature for the 20 hrs. The resultant solution was filtered through Celite, evaporated and purified by silica gel column chromatography (2 g, 230-400 mesh) using 10% methanol in chloroform as an eluent. Yield: 30.4 mg (58%). TLC (CH₂Cl₂/CH₃OH, 4:1): R_{f}=0.48, (CH₂Cl₂/CH₃OH, 9:1): R_{f}=0.18; ATR-FTIR (cm⁻¹): v=3500-3000 (OH, NH₂), 2953 (CH₂), 2923 (CH₂), 2849 (CH₂), 2602 (BH), 1667 (NH₂), 1601, 1581, 1507, 1454 (C=C^{ar}n, 1043 (C—O); ¹HNMR (CD₃OD, ppm): δ=1.480-1.509 (t, 2H, CH₂^{linker}), 1.550-2.850 (m, 10H, B—H), 2.887-2.915 (m, 2H, CH₂^{linker}), 3.220 (s, 1H, C—H^{p-carborane}), 3.861-3.929 (qd, 2H, 5′-H), 4.000-4.021 (q, 1H, 4′-H), 4.294-4.341 (2t, 2H, 2′-H, 3′-H), 6.444-6.452 (d, 1H, 1′-H), 8.313 (s, 1H, 8-H); ¹¹B{H} NMR (CD₃OD, ppm): δ=−18.08 (s), −15.31 (s), −14.66 (s), −13.70 (s), −3.91 (s); ¹³C{H} NMR (CD₃OD, ppm): δ=40.18 (C^{linker}) 63.62 (C-5′), 65.97 (C^{p-carberane}), 68.74 (C^{p-carbonrane),} 78.34, 78.62 (C-2′, C-3′), 86.96, 87.28 (C-1′, C-4′), 118.83 (C-5), 143.19 (C-8), 152.43 (C-4), 157.98 (C-2), 169.04 (C-6); MS (FAB, Gly): m/z=436.4 [M−1]⁻, calc. for C₁₄H₂₇B₁₀N₅O₄ =437.7.

### EXAMPLE 12

### Synthesis 5′-monophosphates 9, 10, 11 and 12

Yoshikawa's procedure for unprotected nucleoside phosphorylation with phosphorus oxychloride was used (Yoshikawa M, Kato T, Takenishi T. A novel method for phosphorylation of nucleosides to 5′-nucleotides. Tetrahedron Lett. 1967, 50, 5065-5068). Suitable nucleoside (0.1 mmol) was dissolved in freshly distilled triethyl phosphate (1 mL). The resulting solution was cooled to 0° C. and further POCl₃ (23 μL, 0.25 mmol) was added. Then the mixture was stirred at 0° C. The reaction progress was monitored by TLC (isopropyl alcohol/water/aq. ammonia, 7:1:2). After completion of the reaction (usually 1.5-2 h), 1M triethylammonium biscarbonate buffer (TEAB, pH 7.5) was added (2 mL) and the mixture was concentrated by evaporation. The crude product 2 was purified by FPLC using a HiPrep 16/10 DEAE FF column (Et₃HN⁺ form,)Pharmacia® equilibrated with TEAB. Chromatography was performed with a linear gradient of TEAB from 0.05M to 0.1M TEAB. The fractions containing the product were combined, concentrated under vacuum and then co-evaporated with ethanol (3×3 mL) to remove traces of buffer. 9: TLC (isopropyl alcohol/water/aq. ammonia, 7:1:2): R_{f}=0.25; UV-vis (95% C₂H₅OH, nm): λₘᵢₙ=229.4, λₘₐₓ=238.5; ATR-FTIR (cm⁻¹): v=2604 (BH); ³¹P NMR (D₂O, ppm): δ=0.51; MS (FAB, Gly, -VE): m/z (%)=513.5 (100%) [M]⁻, calc. for C₁₄H₂₄B₁₀N₅O₇P=513.45; 10: TLC (isopropyl alcohol/water/aq. ammonia, 7:1:2): R_{f}=0.35; UV-vis (95% C₂H₅OH, nm): λₘᵢₙ=231.4, 245.5, 270.3, λₘₐₓ=238.3, 252.5; ATR-FTIR (cm⁻¹): v=2614 (BH); ³¹P NMR (D₂O, ppm): δ=0.31; MS (FAB, Gly, -VE): m/z (%)=497.4 (40%) [M−2]⁻, calc. for C₁₄H₂₅B₁₀N₅O₆P=498.46; 11: TLC (isopropyl alcohol/water/aq. ammonia, 7:1:2): R_{f}=0.27; UV-vis (95% C₂H₅OH, nm): λₘᵢₙ230.0, λₘₐₓ=238.3; ATR-FTIR (cm⁻¹): v=2605 (BH); ³¹P NMR (D₂O, ppm): δ=0.09; MS (FAB, Gly, -VE): m/z (%)=513.3 (100%) [M]⁻, calc. for C₁₄H₂₄B₁₀N₅O₇P=513.45; 12: TLC (isopropyl alcohol/water/aq. ammonia, 7:1:2): R_{f}=0.28; UV-vis (95% C₂H₅OH, nm): λₘᵢₙ=233.9, λₘₐₓ=251.8; ATR-FTIR (cm⁻¹): v=2603 (BH); ³¹P NMR (D₂O, ppm): δ=0.1; MS (FAB, Gly, -VE): m/z (100%)=516.5 (100%) [M−1]⁻, calc. for C₁₄H₂₈B₁₀N₅O₇P=517.48.

1. A nucleoside derivative containing a boron cluster connected with the nucleoside structure at position 2 or 8 of purine nucleobase through a connecting group and its 5′ phosphate, thiophosphate or phosphonate derivative for use as a drug.

2. A nucleoside derivative for use according to claim 1 characterised in that the connecting group is defined by the formula:
(CH₂)ₘWₖ(CC)ₙT₁(CH₂)ⱼQᵢ(CC)ₚ(CH₂)_{r—} where: m, j and r are whole numbers from 0 to 3; k, n, l, i and p each have a value of 0 or 1; W, T and Q independently of one another denote O, S, C(O), S(O), S(O)₂, Se, NR, where R denotes: H, alkyl, haloalkyl, alkoxyalkyl or aryl,
the boron cluster is 1,2-dicarba-closo-dodecaborane (ortho-carboranyl), 1,7-dicarba-closo-dodecaborane (meta-carboranyl), 1,12-dicarba-closo-dodecaborane (para-carboranyl), 7, 8-dicarba-nido-undecaborane (nido-carboranyl), closo-dodecaborane or their derivatives substituted on a carbon or boron atom,
that the nucleoside part of the agent is selected from a group containing a combination of guanine, adenine or cytosine and ribose, deoxyribose or arabinose residue.

3. A nucleoside derivative for use according to claim 1, characterised in that it is the compound defined by the formula 1: 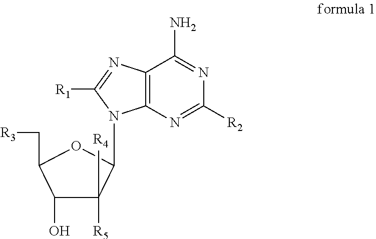
where:
R1 and R2 denote: H or a group with the formula:
(CH₂)ₘWₖ(CC)ₙT₁(CH₂)ⱼQᵢ(CC)ₚ(CH₂)ᵣ— boron cluster
where: m, j and r are whole numbers from 0 to 3; k, n, l, i and p each have a value of 0 or 1; W, T and Q independently of one another denote O, S, C(O), S(O), S(O)₂, Se, NR, where R═H, alkyl, haloalkyl, alkoxyalkyl or aryl, and the boron cluster constitutes a compound selected from a group encompassing: 1,2-dicarba-closo-dodecaborane (orto-carboranyl), 1,7-dicarba-closo-dodecaborane (meta-carboranyl), 1, 12-dicarba-closo-dodecaborane (para-carboranyl), 7,8-dicarba-nido-undecaborane (nido-carboranyl), closo-dodecaborane or their derivatives substituted at the carbon or boron atom. R3 denotes XP(Z)(Y)X¹ where X and X¹ denote: O, S, Se, alkyl, haloalkyl, alkoxyalkyl, aryl, CH═CH, CC, N═N, CHOH or CHN₃; Z denotes: O, S, Se; Y denotes: OH, SH, SeH, H, alkyl, haloalkyl, alkoxyalkyl, aryl, a fluoride atom, particularly fluorine, CH═CH₂, CCH, N═NH, CHOH or CHN₃,
R4 and R5 denote: H, OH, F, N₃, SH, Cl, Br, J, NHR or ¹⁵NHR where R denotes: H, alkyl, haloalkyl, alkoxyalkyl or aryl.

4. A nucleoside derivative for use according to claim 3, characterised in that R3 denotes: OH or O (PO₃)m where m is a whole number from 0 to 3, and R4 and R5 respectively denote: H and OH or OH and H.

5. A nucleoside derivative for use according to claim 1, characterised in that it is a compound selected from the group encompassing:
2-ethynyl-(1,12-dicarba-c/oso-dodecaboran-2-yDadenosine,
8-ethynyl-(1,12-dicarba-doso-dodecaboran-2-yl)-2′ -deoxyadenosine,
2-ethynyl-(1,12-dicarba-doso-dodecaboran-2-yparabinoadenosine,
2-ethyl -(1,12-dicarba-c/oso-dodecaboran-2-yparabinoadenosine or phosphates thereof.

6. A method for treatment or prophylaxis of neoplasms a comprising administering the nucleoside derivative of claim 1.

7. A compound selected from a group encompassing:
2-ethynyl-(1,12-dicarba-c/oso-dodecaboran-2-yparabinoadenosine,
2-ethyl-(1,12-dicarba-doso-dodecaboran-2-yparabinoadeno sine or phosphates thereof.

8. The method of claim 6, where said neoplasm is chronic lymphocytic leukemia.
